(19) United States
(12) Patent Application Publication
(10) Pub. No.: US 2019/0213864 A1
(43) Pub. Date: Jul. 11, 2019

(54) MOBILE DEVICE FINDER

(71) Applicant: Nikia Moree, Norfolk, VA (US)

(72) Inventor: Nikia Moree, Norfolk, VA (US)

(21) Appl. No.: 15/864,552

(22) Filed: Jan. 8, 2018

(51) Int. Cl. G08B 21/24; H04M 1/725; A44C 15/00; A44C 5/00; A61B 5/024

(52) U.S. Cl. CPC G08B 21/24; H04M 1/72527; G08B 7/06; A44C 5/0015; A61B 5/02438; A44C 15/0015

(57) ABSTRACT

The present invention is mobile device finder bracelet which notifies individuals when their mobile devices are not in close proximity to the individual.

### BACKGROUND OF THE INVENTION

### Field of the Invention

The present invention is in the field of consumer electronics. More specifically, the present invention is a mobile device finder.

### Description of the Related Art

The present invention is mobile device finder having bracelet lithium battery gps light bluetooth and a mobile application.

The main purpose of mobile device finder is to present a novel bracelet which notifies individuals when their mobile devices are not in close proximity. Mobile phones are expensive devices which most individuals use daily to store personal information, to access the internet, play games, contact loved ones through social media, text and/or make phone calls, etc., making these electronics one of the most important item in one's possession. However, consumers tend to carry their phones in hand, a habit which is susceptible to loss if set down while busy and a serious concern for forgetful individuals. Ingenious and practical, mobile device finder introduces a bracelet which is able to connect with mobile devices, granting users an immediate alert when they reach a far distance from their phones, preventing occurrences of misplacement. Comprised of a silicone bracelet integrated with Bluetooth™ technology for mobile device connectivity; mobile device finder features light and vibration sensors that activate upon creating a certain distance away from one's cell phone, effectively notifying individuals that the device is not on their possession. mobile device finder additionally offers a free downloadable application which permits users to manage the amount of allowed distance before an alert is provoked, enable a lock status which triggers sensors if an unauthorized person attempts to unlock the phone, as well as a tracker to locate the device if lost. Mobile device finder can easily become an essential mobile device accessory and is intended to be readily available at electronic stores and/or online retailers. Mobile device finder is the only product of its kind to offer a wearable notification accessory to better secure mobile devices. Mobile device finder is uniquely designed to potentially provide a second bracelet for children, empowering parents to utilize the tracking feature to monitor a child's whereabouts via the mobile app, an appealing concept for added versatility.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a mobile device finder, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

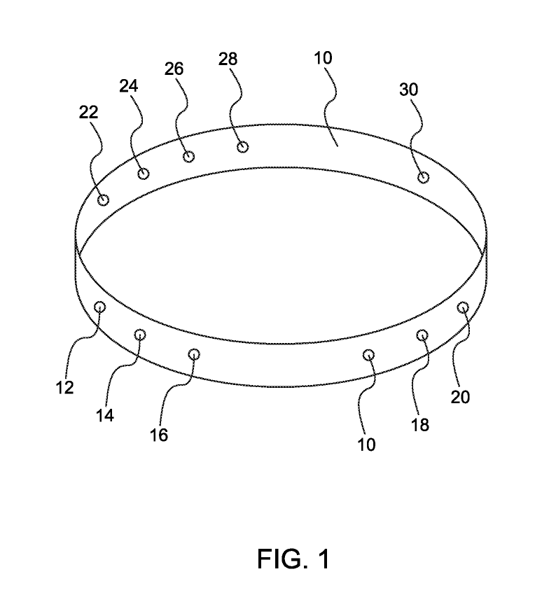

Referring now to FIG. 1, illustrated is a mobile device finder, in accordance with one embodiment of the present invention. In FIG. 1, illustrated is a mobile device finder or water proof bracket 10. All features are on the circular part of the mobile device finder or water proof bracket 10 shown is a lithium battery 12, GPS 14, vibration 16, lights 18, Bluetooth to connect to the mobile device through the mobile device app 20, internet access 22, SOS signal 24, network conductivity 26, sensor for heart rate or pulse 28 and digital signal indicator 30.

While the present invention has been related in terms of the foregoing embodiments those skilled in the art will recognize that the invention is not limited to the embodiments described. The present invention may be practiced with modification and alteration within the spirit and scope of the appended claims. Thus, the description is to be regarded as illustrative instead of restrictive on the present invention.

1. A mobile device finder to find a mobile device via a mobile application running on the mobile device, the mobile device finder comprising:
a wearable bracelet constructed from a flexible material;
a GPS disposed within the bracelet, the GPS having a vibration indicator and indicator lights that activate in response to the mobile device being distanced from the mobile device finder; and
a conductivity system disposed within the bracelet to connect the mobile device finder to the mobile device via the mobile device application running on the mobile device, such that the GPS, the vibration indicator, the indicator lights, and the conductivity system are all disposed within a boundary of the bracelet.

2. The mobile device finder according to claim 1, wherein the mobile device finder is a water proof bracket.

3. The mobile device finder according to claim 1, wherein the conductivity system is a Bluetooth system.

4. The mobile device finder according to claim 1, wherein the conductivity system is internet access.

5. The mobile device finder according to claim 1, wherein the conductivity system is a cellular network.

6. The mobile device finder according to claim 1, wherein the mobile device finder has a SOS signal.

7. The mobile device finder according to claim 1, wherein the mobile device finder has a sensor for heart rate or pulse.

8. The mobile device finder according to claim 1, wherein the mobile device finder has a digital signal indicator.

9. The mobile device finder according to claim 1, wherein the circular part is a bracelet.
